(19) United States
(12) Patent Application Publication
(10) Pub. No.: US 2021/0153773 A1
(43) Pub. Date: May 27, 2021

(54) DEVICE AND METHOD FOR DETECTION AND MONITORING OF COUGH

(71) Applicants: Edward T. Wei, Berkeley, CA (US); Bin Sun, El Cerrito, CA (US); Zhengrong Tang, Sunnyvale, CA (US)

(72) Inventors: Edward T. Wei, Berkeley, CA (US); Bin Sun, El Cerrito, CA (US); Zhengrong Tang, Sunnyvale, CA (US)

(21) Appl. No.: 16/854,641

(22) Filed: Apr. 21, 2020

Related U.S. Application Data
(60) Provisional application No. 62/974,276, filed on Nov. 22, 2019.

(51) Int. Cl. A61B 5/08; A61B 5/00; A61B 7/00; A61B 5/01; A61K 31/194

(52) U.S. Cl. CPC A61B 5/0823; A61B 5/7282; A61B 5/4884; A61B 5/113; A61B 5/01; A61B 5/6823; A61K 31/194; A61B 7/003

(57) ABSTRACT

Cough is a common experience and the most frequent reason why an individual seeks a visit to a physician. The prevalence of cough is about 10+% of the population. Cough is a manifestation of many aerodigestive tract disorders and especially consequential for serious lower airway diseases such as respiratory infections, chronic obstructive pulmonary disease (COPD) and asthma because increased coughing leads to emergency room visits and hospitalization. There is a need for methods to oversee coughing frequency in certain patients. Traditionally, all automated cough monitors have used cough sound as the signal for the measurement of cough. In the present invention movements of the diaphragm muscle, recorded by a motion sensor placed above the xiphoid process, are used for counting coughs. A device for such recordings is described and data were collected. This method is validated by using citric acid spray to trigger cough sounds and to show that the provoked acoustic signal is matched by the electronic signals from the movement sensors. The xiphoid process is a unique anatomical landmark for the non-acoustic detection of cough.

This application claims the benefit of provisional application, U.S. 62/974,276 filed Nov. 22, 20219.

### BACKGROUND OF THE INVENTION

The present invention generally relates to the field of monitoring bodily functions. Continuous monitoring of bodily functions has been facilitated by advances in technology for sensors and for transmission of electronic information from sensors to remote receivers. For example, the use of the Apple watch to record heart rate and to record the number of steps taken per day is now common. Devices for attaching to garments to measure breathing rate have been described by Guay et al. (Sensors 17: 1050-1063, 2017) and by Spire (US 2019/0223799). The use of devices to measure cardiopulmonary function to aid the healthy in improving fitness and athletic performance is now familiar. Devices for such monitoring have also been described (Keenan and Coyle, Method and system for processing data from ambulatory physiological monitoring. U.S. Pat. No. 9,277,871). Medical management of ambulatory patients can also be improved if data can be collected and remotely accessed, for example away from the immediate clinic and the hospital. Medical management, however, requires a more precise and accurate discriminatory selection of collected data because the data will be used for clinical assessment and may impact the outcome of a medical condition.

Cough (and the urge to cough) is a common experience and the most frequent reason why an individual seeks a visit to a physician. Cough is an episodic event manifested in many aerodigestive tract disorders. Cough is designed to clear the airways of actual or perceived obstruction. Short-term cough (acute cough) is caused by viral infections such as the common cold or the flu. Chronic cough (8 weeks) is caused by airway inflammation and conditions such as post-nasal drip and allergy, cough variant asthma, psychogenic disturbances, and acid reflux. Serious lower airway blockage diseases such as chronic obstructive pulmonary disease (COPD) and asthma also increase coughing. The number of coughs per hour vary according to disease: in the healthy it is <1 cough per hour, in asthma (other than cough variant asthma) it may average 3 to 5 coughs per hour, in idiopathic lung fibrosis it may go to 7 to 10 coughs per hour, and higher in chronic cough. In chronic refractory cough, patients may have >40 coughs per hr and this condition lasts for more than 8 weeks. Cough counts before going to sleep at night may exceed 100 coughs per hour. In smokers with chronic obstructive lung disease (COPD), the average was ˜9 coughs/h (n=68) versus 0.7 cough/h for healthy volunteers.

No new medication for cough has been introduced in the past fifty years but new drug candidates for chronic cough, such as P2X3 antagonists, are now in an advanced stage of clinical testing. The prevalence of cough is about 10+% of the population. Cough affects people's lives and diminishes its quality. The US FDA has banned use of codeine by children and restricts adult use, thus creating pressure for new alternatives. It is estimated that over 60 million people suffer from chronic cough in the USA and Europe.

A cough is a reflex clearance mechanism to get rid of unwanted particles or liquids in the airways and the cough is not driven by changes of gas tensions in blood. A cough is audible and episodic, unlike breathing which is quiet, regular, and must be continuous. Unlike breathing, a cough is not a respiratory event of gas exchange. A cough is a co-ordinated muscular effort involving inspiration, closure of the glottis, compression of the air inside the lungs, and rapid opening of the glottis and expiration. An effective cough can generate an airflow of 200+ miles per hour and volumes of 12 L/sec. The mechanism of a loud cough sound is like the popping of a champagne cork. Cough clears the airways of secretions and particles, but the efforts of coughing can also be driven by psychogenic disorders and be non-productive, cause pain, for example, of the rib cage, and the cough can make the throat lining hypersensitive to innocuous stimuli. If cough is controlled patients can sleep better at night.

There is a second type of physiological response, called the expiration reflex, which is similar to the cough reflex. It is driven by laryngeal irritation but the cough muscular responses and sound occurs without an inspiratory phase (Widdicombe et al. Europ. Respir. J. 28: 10-15, 2006).

The technology for developing modern cough frequency monitors was reviewed by Smith and Woodcock (International Journal COPD 2006:1(3) 305-314) and more recently by Shi et al. (J Sensors 2018 Article ID 9845321). So far, the commercial design of cough monitors (for detection of cough frequency) has been based on measuring the acoustic signal generated by cough. Thus, a microphone is used to detect the sound produced by the cough, and the audio signal is transmitted and recorded for further analysis. Techniques using impedance plethysmography or electromyography were suggested, but have not been validated and put into practice (see Smith and Woodcock, 2006). Two cough monitoring systems based on an acoustic signal and demonstrating good validity are now used in clinical trials: the Leicester cough monitor (LCM) and the VitaloJak. They differ mainly in their approach to the analysis of the cough data; the VitaloJak requires manual assessment of condensed cough recordings, and the LCM is largely automated (Spinou and Birring: An update on measurement and monitoring of cough: what are the important study endpoints? J Thorac Dis 2014: 6(S7): S728-S734). A photo showing these systems are in the article by Shi et al. (vide supra). Other monitoring systems were described by Coyle et al. Systems and methods for monitoring cough. U.S. Pat. No. 7,727,161, and Odame and Amoh(US 2018/0199855 Wearable system for autonomous detection of asthma symptoms and inhaler use, and asthma management), but again the detection has been based on sound. In 2005, Coyle et al. described a wearable shirt system for quantifying cough based on respiratory inductive plethysmography (Cough 2005; 1:3, August 4). The basis for measurement and signal detection was electropotential changes recorded as an electrooculogram (EOG), also known as an electromyogram (EMG). In an EMG, recording electrodes (needles) are inserted into the skin and into the muscle to record electrical activity. The electrodes are attached to wires which in turn are connected to monitors. The technology described by Coyle et al. was not pursued.

A convenient, reliable monitor of cough frequency, relatively inexpensive and easy to use, and without wire attachments, could have considerable clinical value. In the pharmaceutical arena, the monitors could be used to evaluate new anti-tussive agents for efficacy. In clinics for respiratory medicine, it can be used for the diagnosis of respiratory infections, and the intensity of airway irritation. The monitors could be used for quantifying the cough hypersensitivity syndrome. For ambulatory patients h airway disease, information on cough could be recorded and transmitted to the physician in charge to assess the breathing status of patients. This information could then be used for decision-making in the admission of patients with airway blockage disease into emergency rooms or intensive hospital care. The admission events, called exacerbations (a worsening of respiratory function), are significant economic burdens to the health care system. The costs per exacerbation can exceed $10,000 per incident per patient with chronic obstructive pulmonary disease, and frequent exacerbations are linked to the rapid death of the patient.

### BRIEF SUMMARY OF THE INVENTION

In this discovery, the sudden acceleration force (measured in G units) generated by the motion of the xiphoid process during cough is the basis for monitoring the cough frequency. The xiphoid process is part of the sternum (breastbone). The sternum is shaped like a sword, with the handle (manubrium) at the rostral end of the ribcage, the middle part of the blade is called the gladiolus, and the tip of the sword is the xiphoid process. The xiphoid is usually triangular, with average dimensions of 1.8 to 2.4 inches (5 to 6 cm) in length, 0.8 to 0.92 inches (2.2 to 2.4 cm) wide, and 0.25 to 0.35 inches (0.73 to 0.82 cm) thick. The xiphoid is connected to the gladiolus at the xiphisternal joint and its motion is more flexible than the sternum. The xiphoid is connected posteriorly by tendons to the diaphragm muscle. The diaphragm is like a piston. In inspiration, the muscle contracts and the piston moves downwards, expands the rib cage, and draws air into the lungs. In expiration, the diaphragm piston moves upwards, and together with the relaxation of the rib cage, forces air rapidly past the larynx. The diaphragm is attached directly to the xiphoid process, and also to the costal cartilage of the ribs, and the lumbar vertebrae. The movements of the diaphragm and ribcage during cough are synchronized, amplified, centralized, concentrated, and transduced to the xiphoid. We discovered that the xiphoid is an anatomical landmark uniquely specific and sensitive to forces generated by cough. The xiphoid moves violently during cough, like a hung towel flapping in the wind, but it is only its movements in the anterior-posterior direction that create the optimal signal for the detection of cough. We propose a device and a method for quantifying cough based on recording the violent accelerated movements of the xiphoid process during cough.

In one aspect of the invention, a device for quantifying cough function and cough dysfunction in a subject in need of such quantification is provided which comprises the use of a motion sensor and a transmitter unit for transmitting the motion sensed by the motion sensor, which device, when attached and positioned on the skin of the subject above the xiphoid process, is adapted to detect, record, and transmit signals of accelerated movements of the xiphoid. Thus, an accelerometer motion sensor positioned on the skin above the xiphoid process may be used to record the 3-dimensional movement of the diaphragm muscle during cough. The sensor unit, precisely positioned with antennae in the fabric overlying the skin, can transmit the motion signals in multiple coordinate axes to a distal unit for recording and further analysis. The signal to be detected is an acceleration of mass or G units of xiphoid process, in particular in the anterior-posterior direction, and not a change in the volume or other parameters of the rib cage.

In another aspect of the invention, a method is provided for quantifying cough function and cough dysfunction in a subject in need of such quantification comprising:
providing a motion sensor and a transmitter unit for transmitting the motion sensed by the motion sensor; said motion sensor and transmitter unit on the skin of the subject above the xiphoid process; and, recording, and transmitting signals of xiphoid process movements.

This method does not require measurement of the cough sound, or thoracic muscle activities, or changes in the thoracic or abdominal volumes. Optimized placement of the accelerometer sensor above the xiphoid landmark is important: for example, placement of the sensor on the skin over the pectoral muscles or the abdominal muscles does not generate a clear signal. The major relevant cough signal that is detected and recorded is the sudden episodic and violent movement of the xiphoid in the anterior-posterior axis.

This cough measurement system from the xiphoid is demonstrated and validated using a citric acid spray as a tussive stimuli. When citric acid, dissolved 50 mg/mL in distilled water, is placed in a perfume sprayer and held about a foot away from the nose, activation of the nozzle releases a fine spray of ˜0.07 to 0.1 mL which, when inhaled, instantly causing a brief bout of one to six coughs. Simultaneous recordings of the cough sound and the transmitted signal from the sensors on the xiphoid process validate the test unit. The use of the citric acid spray to mimic pathological cough is part of the inventive steps in this discovery.

### BRIEF DESCRIPTION OF THE SEVERAL VIEWS OF THE DRAWINGS

FIG. 1. is an illustration of a spray bottle containing a citric acid solution (50 mg/mL in water). The nozzle is pointed at and held about a foot away from the nose. Activation of the sprayer by one or two pumps will initiate a tussive stimuli and the resultant cough sounds can be recorded and matched against the movements recorded from the xiphoid. This citric acid challenge, synchronized with the sensors, validates the data from the system.

FIG. 2. shows the device for recording movements of the xiphoid process. The antennae for the device are in the fabric placed above the skin and focused on the center of the chest, over the xiphoid process. The principal components in this device are the mechanical elements, the sensing mechanism, and the application-specific integrated circuit (ASIC). The recorded signals are stored in a flash drive and transmitted wirelessly to a receiver for processing.

FIG. 3. shows the topographical reference points for quantifying the movement of the xiphoid process. The x-axis is in the rostral-caudal direction, the y-axis is in the medial-lateral direction, and the z-axis is in the anterior-posterior direction. The xiphoid process is the key reference point for monitoring cough because the xiphoid process is tightly coupled to and amplifies the movements of the diaphragm. The z-axis gives a very clear signal response to cough.

FIG. 4. is an illustration of the movement of the xiphoid/diaphragm in the XYZ axes after a tussive challenge with citric acid. The accelerometer tracings shown here are synchronized with a microphone-recorded cough sounds of a male subject exposed to the citric acid spray. The z-axis movement gives an excellent clear signal of cough. The x and y axes signals can give false signals. The point of origin of the cough sound is marked as dashed black squares.

FIG. 5. is an illustration of the movement of the xiphoid in the XYZ axes after a tussive challenge with citric acid. The gyroscope tracings are synchronized with a microphone-recorded cough sounds of a male subject exposed to the citric acid spray. The y-axis gives a point of reference for the position of the body in space. The point of origin of the cough sound is marked as dashed black squares. It can be seen that angular velocity measurements can give false signals of cough.

FIG. 6. is a magnified illustration of the specific movement of the xiphoid in the z-axis after a tussive challenge with citric acid. The z-axis movement of the xiphoid gives a robust, powerful, clear and unique signal of cough, and which is not replicable by breathing or physical movements.

### DETAILED DESCRIPTION OF THE INVENTION

Diaphragm Muscle and Xiphoid Process.

To breathe and stay alive we continuously contract and relax the thoracic and abdominal muscles for breathing about 30,000 times per day. This marathon activity of inspiration and expiration is performed by the diaphragm, the abdominal muscles, and the muscles of the rib cage. The diaphragm (Greek, meaning “partition”) is a dome-shaped muscle that separates the thorax, or chest, from the abdomen. During inspiration, the diaphragm contracts and pulls downward while the muscles between the ribs contract and pull upward. These movements increase the size of the thoracic cavity and decrease the pressure inside. As a result, air enters and fills the lungs. The diaphragm is the most important muscle used in inspiration. During exhalation, the rib cage and chest wall passively start to sag and revert to the original position. At the same time, there is relaxation and elevation of the diaphragm. This motion forces the air within the lungs to push out of the body. Normal breathing is rhythmic, even during exercise, and is quantified by volume displacement of air and the accompanying movement changes in the rib cage and abdominal cavity. By contrast, a cough is an exaggerated, violent and explosive event, with G forces generated in bone movements. Severe cough in the elderly can fracture ribs. The cough is designed to expel real or perceived obstruction in the airways. The cough has imperative survival value and urgency.

The diaphragm is attached to the body's structures by ligaments and tendons. One important diaphragm attachment is to the xiphoid process, a small triangular blade-tip like structure at the base of the breastbone (sternum), in the center of the chest. The xiphoid is usually triangular, with average dimensions of 1.8 to 2.4 inches (5 to 6 cm) in length, 0.8 to 0.92 inches (2.2 to 2.4 cm) wide, and 0.25 to 0.35 inches (0.73 to 0.82 cm) thick. The xiphoid is hyaline cartilage at birth and slowly becomes bony (ossified) at about the age of 40. It is attached to the body of the sternum at the xiphisternal joint. Muscles attached to the xiphoid also include the rectus abdominis and the transverse abdominis. The intercostal muscles and the ribs are attached to the breastbone. The diaphragm is the primary muscle for moving air. Its excursive movements during cough is tightly coupled to the movement of the xiphoid and can be felt by placing a finger over the xiphoid. Surprisingly, there has not been a clear recognition of the value of measuring cough frequency based on automated recording of the violent characteristic movements of the xiphoid in the anterior-posterior axis during cough. The movement of the xiphoid has not been explicitly identified as a source of landmark signals for wireless cough recordings. Instead, the focus on cough measurements has always focused on the acoustic signal.

Sensors and Wearable Recording Antennae

The principal sensors in the device for measuring movements of the xiphoid and accompanying diaphragm are an accelerometer sensor that can detect forces of ±8G along three axes. A gyroscope sensor for the detection of angular movement of 360° may be added for sensing vertical orientation. The antennae for these sensors are in the fabric placed over the recording area, namely, the skin above the xiphoid process above the center of the chest. In addition to these sensors, more sensors may be incorporated into the array such as vibration sensors to measure chest wall oscillations, and a MEMS microphone sensor array to corroborate the audio changes in cough function. A temperature sensor may also be incorporated to detect fever. The primary and necessary sensor is, however, the linear accelerometer to detect motion in the z-axis.

Tussive Challenge and Validation of Device with Citric Acid Spray

Citric acid is an organic acid found naturally in citrus fruits. It occurs as a white crystal that is soluble in water at up to 540 mg/mL. Citric acid has been used as a tussive agent in the investigation of the cough reflex (Wong et al. Cough induced by low pH. Respiratory Medicine 93: 58-61, 1999). In these studies, citric acid solutions were delivered using a nebulizer or an inhaler. When applied in this manner, coughing was observed. The mechanism of action of cough induced by citric acid is caused by acid irritation of upper airway (nasal, pharyngeal and tracheal) membrane nerve endings.

Here, a citric acid (BulkSupplements.Com. 7511, Eastgate Road, Henderson, Nev. 89011, ˜100% pure) solution is prepared at 50 mg/mL, dissolved in distilled water, and put in a perfume sprayer. When held at about 1 foot in front of the nose and activated, a fine spray of ˜0.07 to 0.10 mL is released per activation. This spray when inhaled will instantly provoke one to six coughs in every subject tested (n=10) in less than several minutes. The cough sound is synchronously recorded with the tracings obtained from the sensors attached to the xiphoid process to show that the two events are tightly coupled and correlated. This is a method to validate the recordings made by the motion sensors. The citric acid challenge can also be a method to provoke a standardized muscle response such as may be used for measuring lung compliance (a measure of the lung's ability to stretch and expand, that is, the distensibility of elastic tissue). The normal breathing rhythm is variable and does not give a sufficient excursion to give a clear diaphragm signal/noise recording for measuring lung compliance. Thus, a designed citric acid challenge method for this application is an innovative experimental tool when linked to a wireless recording device.

Clinical Use of a Cough Monitor in Cough Function and Dysfunction

An automated, inexpensive cough monitor, with antennae sensors, positioned over the skin above the xiphoid process, and sending signals to a remote data storage facility, has many potential applications. For example, it can be used in clinical trials to evaluate the efficacy of new anti-tussive candidates. In the management of patients with cough hypersensitivity syndrome. A cough monitor may be used to identify stimuli that trigger cough and determine the status of a subject's selective sensitivity to tussive stimuli. For example, it may be used to differentiate a subject's sensitivity to perfumes by contrast to sensitivity from pollens, and thus aid in diagnosis. Cough frequency provides a quantitative dimension for the physician's assessment. Cough counts may also help a physician identify respiratory infections and the illness status of a patient with asthma or lower airway blockage diseases such as COPD. For example, cough counts in an asthmatic together with oximeter readings may help determine if a subject is under a real or imagined threat of hypoxia and requires emergency services. Most exacerbations of respiratory diseases are precipitated by viral or bacterial respiratory infections. An objective cough count will help confirm the severity of a lung condition, e.g. by showing increased cough counts during an infectious exposure. A high-frequency cougher may also be a “super-spreader” of infectious agents and a cough monitor can help identify such an individual. Eventually, a reliable automated cough count may become a diagnostic and monitoring tool for the pulmonary physician just as a heart rhythm recordings are useful for the cardiovascular specialist.

Below are definitions and descriptions that may further help illustrate the discovery.

Cough. Cough (and the urge to cough) is a common experience. Cough stimuli can enter the throat by breathing in, e.g. smoke and nasal secretions, or be initiated by stimuli wafted up into the throat from the airways, e.g. phlegm. Each cough involves co-ordinated synchronized muscular effort. An effective cough can generate an air velocity of 280 m/sec and volumes of 12 L/sec. Cough clears the airways of secretions and particles. But, cough can also be non-productive (dry and hacking), painful to the throat, and exhausting because of increased muscular effort. The throat lining can become hypersensitive to innocuous stimuli. If patients are taught to control cough they can sleep better at night and this control may be utilized to clear mucus. Chronic cough may be due to subtle throat irritation from acid reflux. It can also be a psychogenic disorder, like habitual nervousness or a tic. Management of cough in an important clinical event because the cough is so common. Thus, a device to provide a quantitative index of cough has utility for diagnosis and patient management.

Cough as an Index of Pathological Events. Cough is about removing the real or perceived obstruction in the airways. Normally, the airways are used for breathing which is about bringing oxygen to the blood and removing carbon dioxide. The two events are separate and distinct. Many new applications can be envisioned if cough frequency can be easily and selectively measured for the patient. For example, the smoker may be warned by an increased cough count that the airways are being damaged and be encouraged to quit. If cough increases, lung cancer may be present and detected. Cough can be stimulated by irritants on the upper airways without the overt presence of obstruction. For the patient with allergic cough, an objective increase in count may suggest that the anti-inflammatory medications for rhinitis are not working and should be modified. This will also apply to the asthma patient with a cough. For acid-reflux induced cough, again an objective cough count may help guide therapy. The common cold and influenza viruses affect the upper airways. In the US, there are about 1 billion cases of upper respiratory tract infections per year. Post-infectious upper respiratory tract cough is quite common, but this event is not well quantified or studied. A cough monitor will be an important research tool for this subject. In many patients with chronic refractory cough (8 weeks of coughing), there is no evidence of airway obstruction. In such patients, a good cough count will help determine if psychogenic factors are causing the cough. The irritative signals in the throat received by the brain are under voluntary control. The amplification of such signals in perception may be influenced by stress. Thus, the use of a cough in chronic refractory cough may reveal psychiatric disturbances. There are many applications for a cough monitor in clinical practice because it provides an objective quantitative index of bodily functions.

The electrocardiogram (ECG or EKG) is a standard measurement of cardiac muscle depolarization and repolarization. As such, it provides instantaneous information on cardiac rhythms. Cardiac output, on the other hand, is a measure of the ability of the heart to pump blood. For the lungs, the tidal volume and FEV₁ are analogous to cardiac output, but for airway obstruction, there are no immediate readouts of functional status. Measurement of cough frequency provides valuable information on the immediate state of actual or perceive airway blockage. Eventually, the cough frequency (C_{f}) may become a standard index of airway hygiene.

Cough Monitor and Transmission of Viruses Viruses are transmitted between individuals by direct contact, such as exchange of body fluids, by contacting the opposite party with inanimate contaminated surfaces called fomites, and by airborne inhalation of or mucous membrane contact (eyeball) with airborne droplets containing the virus. All of these routes of transmission have been shown with viruses such as the AIDS virus, influenza virus (swine and avian), and beta coronaviruses (CoV), such as the SARS, MERS, and Covid-19 viruses. Transmission via the airborne droplets route appears most common, although not proven. A famous case was described by Gregg (The epidemiology of influenza in humans. Annals NY Acad Sciences 353:49-53, 1980). A 21-year old woman with a cough managed to infect 37/52 (71%) of passengers with influenza in the 4.5 hr period when she was inside a stationary cabin of a Boeing 737 airliner. She stayed in the rear seat of the plane and did not have direct contact with passengers, but only coughed.

Viral transmission rate is a key factor in the progress of an epidemic. In the past, the decimation of health care personnel, nurses and doctors, by a SARS or a MERS patient's infection was alarming, as personnel resources are limited. One way to use a cough monitor is to couple it with a temperature sensor and oblige a patient to wear it, so the information is transmitted remotely, to minimize contact with health care personnel. Another way to use the cough monitor is to determine if a person is a “super-spreader”, defined in the SARS episode as a patient that gives the disease to at least 8 other individuals. A patient with a high cough frequency is likely to be a super-spreader. Identifying such individuals will be important for isolation or quarantine because transmission of infection is caused primarily by 20% of the infected population. If this 20% super-spreader population can be identified, the other 80% of the infected does not need to be quarantined.

The cough monitor would also be important for determining the onset of pulmonary pathology and for diagnosis. The incubation time of beta coronaviruses can be up to a week or longer. An objective cough count will help identify and track the course of infection. The cough symptom was manifested in 83% of the patients with SARS in Hong Kong, and 59 to 82% in the current Covid-19 episode in Wuhan, China. For example, in a respiratory disease outbreak or pandemic, the monitor can be used to quantify, in a research setting, the infectivity and tropism of the infective agent. The infectivity of a pathogenic organism is defined as the ability of an agent to cause infection in a susceptible host. A basic measure of infectivity is the number of particles required to cause infection or the number of ill per number exposed. Cough frequency may be a basic parameter of infectivity. For example, a persistent dry hacking cough may predict greater infectivity. Similarly, a deep cough may be indicative of viral tropism for the lower respiratory tract.

Cough Hypersensitivity Syndrome (CHS). CHS was defined by the European Respiratory Society as a condition in which the cough is caused by stimuli that don't usually cause cough, or by a hypersensitivity to stimuli that are known to be tussive, e.g. citric acid or capsaicin. While this hypersensitive mechanism has been imputed initially in patients with chronic cough where no cause of the cough has been found, there is now evidence that even in patients with chronic cough associated with conditions such as asthma, chronic obstructive pulmonary disease, pulmonary fibrosis or gastroesophageal reflux disease, this hypersensitive mechanism underlies the chronic cough. Patients with CHS may have hypersensitivity to stimuli that do not usually induce coughing e.g. talking, laughing, going outside in cold weather or smelling perfume. Other common complaints are a sensation of having something stuck or irritating in the throat, and difficult breathing such as a feeling that there is a blockage at the level of the throat and the patient cannot get air into the lung. Most patients presenting with chronic cough have CHS. A cough monitor will aid in the diagnosis of CHS because an accurate record of coughing can be maintained. The cough monitor can also be used for gauging the sensitivity of a subject to tussive challenges such as capsaicin and citric acid.

Chronic Lower Airways (Lung or Pulmonary) Blockage Disorders. The airways and the lungs, like the heart, brain, liver, and kidneys, are a major organ system essential for survival. Damage to the airways and alveoli surface is quite common, and a widespread cause of human suffering, morbidity, and mortality. For example, chronic obstructive pulmonary disease (COPD) is the third to the fifth leading cause of death in most of the countries in the world. The clinical results of the lower airway blockage disorders are cough, increased sputum production, and dyspnea (in more severe cases), and breathlessness which causes anxiety and panic attacks. Control of cough discomfort relieves patient anxiety and enables the deliberate clearance of accumulated mucus by the patient. With psychic control of throat discomfort, the patient has less anxiety and gets a good night's sleep. Currently, there is no cure for COPD, but the overall management goal is to improve health status, prevent exacerbations, and prevent COPD-related complications and mortality. Any objective method of assessing COPD severity, e.g. cough frequency, will aid in the management of this condition.

COPD is a life-threatening disease. Worldwide deaths from COPD for 2015 were estimated to be 3.2 million individuals and projected to increase. COPD diagnosis is confirmed by spirometry which measures how deeply a person can breathe and how fast air can move into and out of the lungs. The four stages of COPD severity are defined by the FEV₁ value from spirometry (forced expiratory volume in 1 second). COPD is a large burden on the healthcare system. The disease includes conditions called chronic bronchitis, bronchiectasis, bronchiolitis obliterans and emphysema. Chronic bronchitis is an inflammation of the lining of the bronchial tubes. Emphysema is a condition in which the alveoli at the end of the smallest air passages (bronchioles) of the lungs are destroyed. Related obstructed airflow disorders such as asthma and fibrotic lung diseases manifest symptoms similar to COPD. COPD is caused by injury to the lung by agents such as cigarette smoke, chemicals, air pollutants, allergens, and viral and bacterial infection.

Use of Cough Monitors in COPD Exacerbations. COPD is a burden on the health care system because patients require time-consuming care. In countries with well-developed health care, the costs for the advanced COPD patient with exacerbations are hospitalization (71%), prescriptions (19%), and outpatient visits and exams (10%). Generic antibiotics or corticosteroids for treatment cost about 10% of the newer inhaled drugs. A COPD exacerbation (ECOPD, or AECOPD) is defined as “an acute worsening of breathing symptoms that requires additional therapy”. The symptoms that worsen are breathlessness, increased sputum volume, and increased sputum purulence, usually triggered by airway infections or by air pollutants. The definition of “exacerbation” has been thoroughly reviewed by Oca et al. (Medical Sciences 6:1-18, 2018) and these definitions are hereby incorporated by reference. In the ECOPD patient's quality of life, lung function, and life expectancy declined rapidly. For example, in a study population in Italy of 15,857 (average age 76) with ECOPD, 47% died after 29 months. (Blasi, F. et al. The Clinical and Economic Impact of Exacerbations of Chronic Obstructive Pulmonary Disease: A Cohort of Hospitalized Patients PLOS One 9(6):1-8, 2014), In 5 years in the Beijing area, 161,613 hospitalizations for acute exacerbation of COPD were recorded. (Liang, L. et al. Lancet Planet Health. 3(6): 1-18, 2019).

Acute breathlessness is the most terrifying aspect of living with COPD and ECOPD.

Breathlessness creates feelings of urgent need for help. Patients with COPD experience an ongoing cycle of good and bad days. Living with daily breathlessness is a perpetual struggle, and living becomes hard work. Breathlessness is accompanied by fatigue, limited activities and negative mental states, including depression. Daily acts such as sleeping, cleansing, dressing, eating, work, walking, driving, having sex, sports, and talking may be affected. Physicians view COPD as “ . . . inexorable decline: a prolonged period of disabling dyspnea and increasingly frequent hospital admission reflecting deteriorating lung function and usually presaging a premature death.” But the patient may have higher expectations of survival or accommodation to the disease. (Giacomini, M. et al. Ontario Health Technology Assessment Series, 12(13): 1-47, 2012).

The pattern of key COPD symptoms is well-documented. In an electronic Diary survey of 209 patients, completed twice a day (morning and evening) for 26 weeks, the symptoms of shortness of breath, phlegm/mucus, and cough occurred daily in more than 70% of entries. In a questionnaire sample of 2000 patients, breathlessness, phlegm, and cough were reported in 72%, 64%, and 59% of the patients, respectively (Molen T. et al. International J. COPD. 8: 461-71, 2013). Using a 24-hr monitor, COPD current smokers (n=68) had cough rates of 9 coughs/h (median), almost double that of COPD ex-smokers (4.9 coughs/h), whereas healthy volunteers coughed 0.7 coughs/h and healthy smokers had 5.3 coughs/h (Sumner H, et al. Amer J RespCrit Care Medicine 186: 943-949, 2013). The role of cough monitoring in the management of COPD and ECOPD is still in its early stages of development. The potential role of cough monitoring in anticipating and predicting ECOPD may have tremendous significance. Of the major COPD symptoms, only cough can be measured objectively and quantified. Rapid access to information on cough frequency may provide a better understanding of the patient's overall clinical status and guide treatment accordingly. Including a temperature sensor in the device can also give information of fever, which for lung dysfunction is a serious prognosticator of infection.

Asthma is a chronic lower airway disorder characterized by inflammation, wheezing, cough, increased airway resistance, and troubled breathing. Most people have “allergic asthma”, which means that the disease is triggered by allergens. In cough variant asthma, cough is the predominant symptom.

Economic Burden. In both lower airway blockages diseases and asthma, emergency calls and hospital admission events, called “exacerbations” (a worsening of respiratory function), are significant economic burdens to the health care system. The costs per exacerbation can exceed $10,000 per incident. In 2010, the estimated total cost of COPD in the U.S.was $32.1 billion for direct health care costs (Center for Disease Control and Prevention, https://www.cdc.gov/copd/infographics/COPD-costs.html). In the European Union, the annual costs of direct primary and hospital healthcare for respiratory disease was estimated at €55 billion (https://www.erswhitebook.org/chapters/the-economic-burden-of-lung-disease/). Many of the exacerbation (ECOPD) cases are caused by panic attacks and not by direct threat of inadequate ventilation. Thus, methods for improving accurate differential diagnosis of airway status have considerable economic value.

A cough is a “symptom” if the patient notices it and complains. On the other hand, a physician or a nurse can notice the cough and count it, then it becomes a “sign”. If both notice the cough, then it can be either a symptom or a sign. Cough can become an important prognosticator of airway health status if it can be reliably quantified. Then the cough frequency is an objective index of the status of obstruction and this information guides therapy.

Example of Device

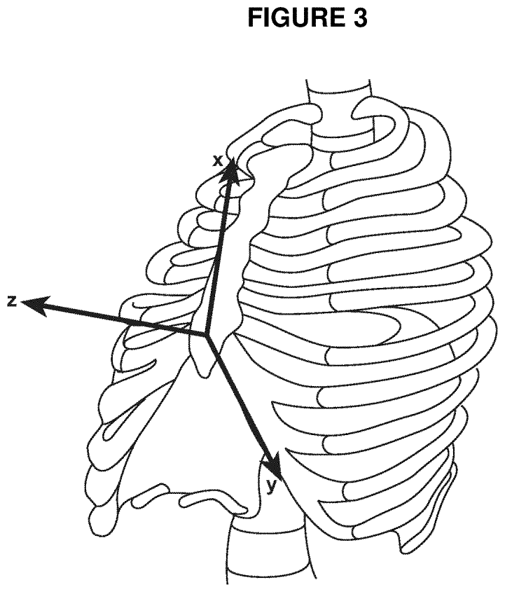

Device sensors for external attachment to the xiphoid process. Accelerometers are devices that measure acceleration, which is the rate of change of the velocity of an object. Accelerometers measure in meters per second squared (m/s²) or in G-forces (g). A single G-force for Earth is equivalent to 9.8 m/s². When coupled to an electronic device, an accelerometer measures linear forces with units of millivolts/g (mV/g). By attaching an accelerometer to an object, its acceleration and gravitational pull in 3-axes, X Y and Z, can be recorded (FIG. 3). A gyroscope gives the rate of change of angular velocity over time with units of mV per degree per second (mV/deg/sec). The gyroscope measures the attached object's angular changes. The accelerometer and gyroscope used in the cough monitoring device are made on a micrometer silicon substrate using micro-electromechanical systems (MEMS). The major components in the device are the mechanical elements, the sensing mechanism and the application-specific integrated circuit (ASIC). All MEMS accelerometer and gyroscope sensors commonly measure the displacement of a mass with a position-measuring interface circuit. That measurement is then converted into a digital electrical signal through an analog-to-digital converter (ADC) for digital processing for storage on a flash drive, and wireless transmittal.

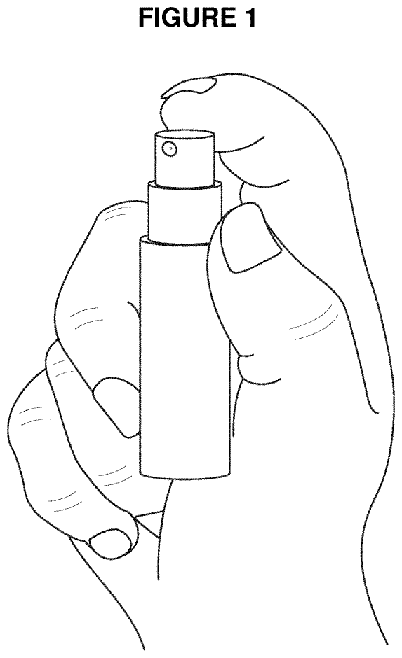

An example of the practice of this invention is shown in Figures (FIG. 1 to FIG. 6).

FIG. 1. is an illustration of a spray bottle containing a citric acid solution (50 mg/mL in water). The nozzle is pointed at and held about a foot away from the nose. Activation of the sprayer by one or two pumps will initiate a tussive stimuli and the resultant cough sounds can be recorded and matched against the movements recorded from the xiphoid. This citric acid challenge, synchronized with the sensors, validates the data from the system.

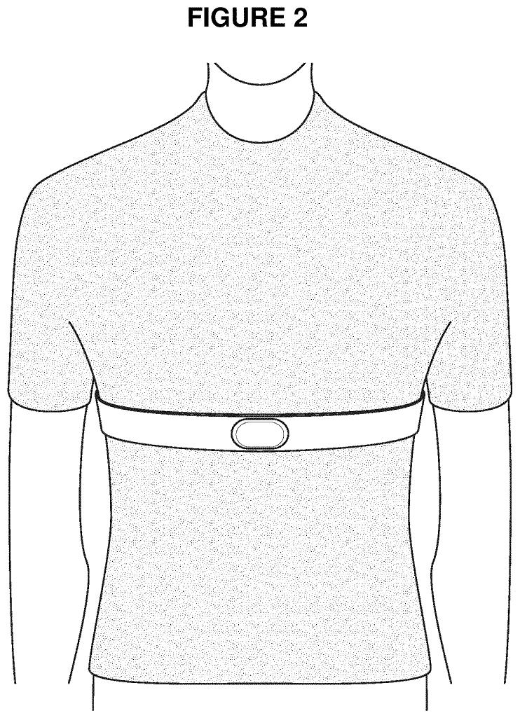

FIG. 2. shows the device for recording movements of the xiphoid process. The antennae for the device are in the fabric placed above the skin and focused on the center of the chest, over the xiphoid process. The principal components in this device are the mechanical elements, the sensing mechanism, and the application-specific integrated circuit (ASIC). The recorded signals are stored in a flash drive and transmitted wirelessly to a receiver for processing.

FIG. 3. shows the topographical reference points for quantifying the movement of the xiphoid process. The x-axis is in the rostral-caudal direction, the y-axis is in the medial-lateral direction, and the z-axis is in the anterior-posterior direction. The xiphoid process is the key reference point for monitoring cough because the xiphoid process is tightly coupled to and amplifies the movements of the diaphragm. The z-axis gives a very clear signal response to cough.

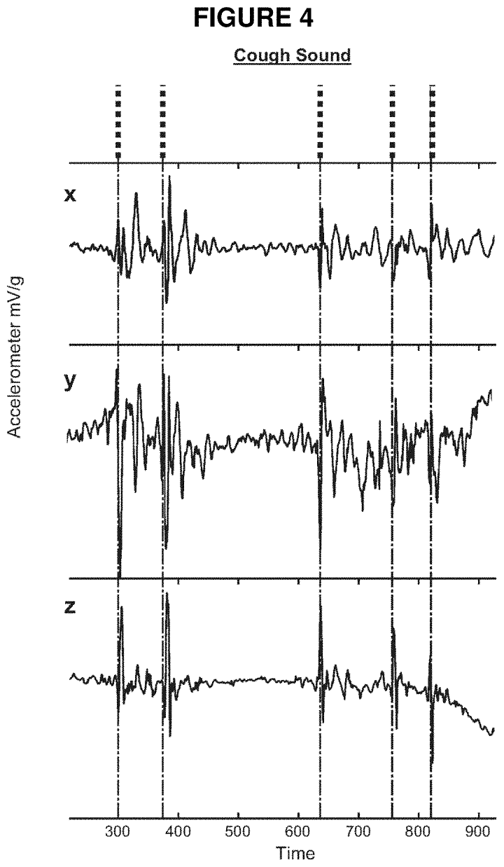

FIG. 4. is an illustration of the movement of the xiphoid/diaphragm in the XYZ axes after a tussive challenge with citric acid. The accelerometer tracings shown here are synchronized with a microphone-recorded cough sounds of a male subject exposed to the citric acid spray. The z-axis movement gives an excellent clear signal of cough. The x and y axes signals can give false signals. The point of origin of the cough sound is marked as dashed black squares.

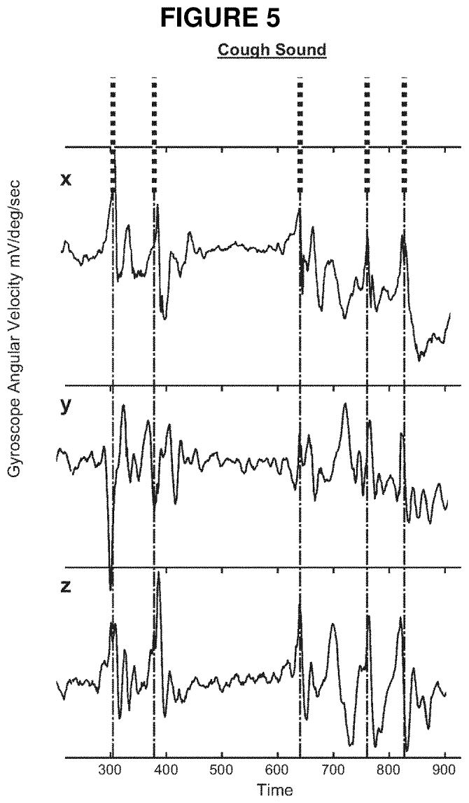

FIG. 5. is an illustration of the movement of the xiphoid in the XYZ axes after a tussive challenge with citric acid. The gyroscope tracings are synchronized with a microphone-recorded cough sounds of a male subject exposed to the citric acid spray. The y-axis gives a point of reference for the position of the body in space. The point of origin of the cough sound is marked as dashed black squares. It can be seen that angular velocity measurements can give false signals of cough.

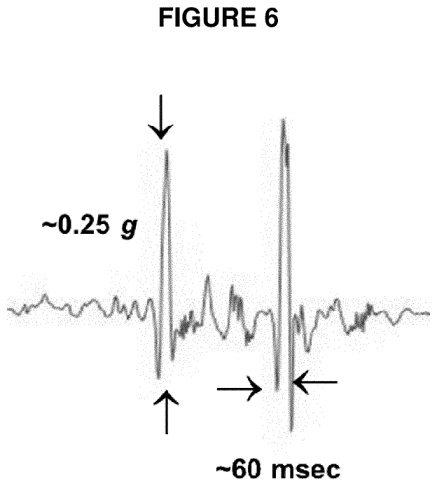

FIG. 6. is a magnified illustration of the specific movement of the xiphoid in the z-axis after a tussive challenge with citric acid. The z-axis movement of the xiphoid gives a robust, powerful, clear and unique signal of cough, and which is not replicable by breathing or physical movements.

A key element of validation of this discovery is the use of the citric acid challenge to identify the XYZ accelerometer signals that correspond to cough. The citric acid spray is a reliable tool for cough provocation.

Cough is an episodic event, unlike respiratory rate or heart rate which are regular and continuous events. Cough requires a different set of electronic signals for detection and discrimination. For example, if the sound is used for monitoring cough, “environmental coughs” (people coughing in the background, see Kulnick et al. J. Thoracic Diseases 8:3152-3159, 2016) are confounding adventitious factors in monitoring. On the other hand, movements of the xiphoid process are uniquely suitable for the individual's motion sensors and will not be confounded by environmental coughs.

From the data in FIG. 4 and FIG. 6, it can be seen that the z-axis signal has the best information content, with a high signal to noise ratio and a direct correlation to the timing of the citric acid cough sound. This is a surprising and unexpected observation. The z-axis signal illustrates the uniqueness of this inventive method of measuring cough. The z-axis cough parameter detects a spasmodic jerk of the xiphoid process in the anterior-posterior direction (z-axis), caused by the movement of the diaphragm. The XY axes can give false signals. Cough cannot be accurately measured by a recording of breathing rate based on circular medial-lateral expansion (y-axis) and contractions rib cage. The cough tracing for the z-axis (FIG. 6), in force and speed, cannot be imitated by breathing. The novelty of the z-axis signal and its weighting can be incorporated into the algorithm for the integrated circuit when it is used to interpret the signals: that is, for specifying the G force range and time scale in msec that defines the cough signal. In the contemplated mode for the practice of this invention, the incoming signals from the sensor are weighted and optimized for the z-axis signal. The gyroscope recordings provide additional information on body orientation and vertical position to verify and validate the recorded signals from the accelerometer but is not essential.

By choosing the correct placement of the sensor, namely, over the xiphoid process, the use of a linear accelerometer is necessary and sufficient for the measurement of cough. A gyroscope accelerometer has value for the spatial orientation of the subject.

A microphone, a pair of myographic electrodes, or a pressure sensor, or any additional type of sensor, is not required.

Experimental Details

The act of coughing causes the device to vibrate and the vibrations are registered by the G sensor (or accelerometer) and Gyro sensor (or gyroscope) integrated in the device. The steps of the procedure are: a) start the device and keep the motion sensors standby, b) start the voice recorder and keep recorder standby, c) capture the coughing signals and record the motions simultaneously for validation, and d) take 5-sec intervals for analysis.

The chest movements during each cough cause vibrations of the device which are registered by the G sensor (or accelerometer) and the Gyro sensor (or gyroscope) integrated in the device. We discovered that the measurements in the direction of the z-axis registered by the G sensor are the most relevant to the cough count. The spike made with each cough has an energy of ≥0.25 G (where 1 G=9.8 m/s2) and occurs in less than 60 msec. The sharp acceleration trace, in a narrow period, is visualized as the spike in FIG. 6.

The type of spike caused by cough cannot be recorded with a normal or even a deep breath because the peak acceleration forces of breathing will be much smaller and the time much longer. Other than coughing, no internal forces within the human body would generate the z-type of spikes in chest movement seen in FIG. 6. The only alternative possibility for such spikes to occur would be an external force or hit against the body, or perhaps sneezing, but in such a situation, the traces for the G and gyro sensors in the x and y axes will differentiate the signals from cough. This is why information on the three axes are collected in the computational model.

Additional tests were conducted, including female subjects, with similar results. The following conclusions were reached: a) The z-axis measurement of the accelerometer is the most sensitive indicator for the detection of cough; b) The gyro sensor measurements are useful for monitoring the scale/range of body movement; c) The y-axis measurements of the accelerometer and gyroscope give useful body position information when combined with measurements on the z-axis; d) This method of measuring cough is not sensitive to gender distinction, and e) Placement of the sensor on the skin above the pectoral muscles, on the lateral rib cage, or on the abdomen does not yield a clear cough signal. To our knowledge, this is the first time that the surprising violent, episodic movement of the xiphoid process during cough has been detected, quantified, and demonstrated.

1. A device for quantifying cough function and cough dysfunction in a subject in need of such quantification comprising:
a motion sensor and a transmitter unit for transmitting the motion sensed by the motion sensor, which device when attached and positioned on the skin of the subject above the xiphoid process is adapted to detect, record, and transmit signals of muscle movements of the diaphragm.

2. The device as in claim 1 wherein the motion sensor is an accelerometer.

3. The device as in claim 1 wherein the cough function that is quantified is cough frequency.

4. The device as in claim 1 wherein the cough function that is quantified is cough intensity.

5. The device as in claim 1 wherein the cough function that is quantified is the lung compliance response to a citric acid spray challenge.

6. The device as in claim 1 wherein the cough function that is quantified is used in the diagnosis of the individual's status as a “super-spreader” of airborne viral particles.

7. The device as in claim 1 wherein the cough dysfunction that is quantified is the altered cough frequency that occurs in infections of the respiratory tract.

8. The device as in claim 1 wherein the cough dysfunction that is quantified is the altered cough frequency that occurs in lower airway blockage disease.

9. The device as in claim 1 wherein the cough dysfunction that is quantified is the altered cough frequency that occurs in chronic refractory cough.

10. The device as in claim 1 wherein the cough dysfunction that is quantified is the altered cough frequency that occurs in the chronic cough hypersensitivity syndrome.

11. The device as in claim 8 wherein the lower airway blockage disease is chronic obstructive pulmonary disease.

12. The device as in claim 8 wherein the lower airway blockage disease is an exacerbation episode of chronic obstructive pulmonary disease.

13. The device as in claim 8 wherein the lower airway blockage disease is asthma.

14. The device as in claim 1 wherein the device has an additional sensor for detecting vibrations of the body.

15. The device as in claim 1 wherein the device has an additional sensor for detecting sounds from the body.

16. The device as in claim 1 wherein the device has an additional sensor for detecting temperature from the body.

17. A method for quantifying cough function and cough dysfunction in a subject in need of such quantification comprising:
providing a motion sensor and a transmitter unit for transmitting the motion sensed by the motion sensor; said motion sensor and transmitter unit on the skin of the subject above the xiphoid process; and, recording, and transmitting signals of diaphragm muscle movements.

18. The method as in claim 17 wherein the motion sensor is an accelerometer.

19. The method as in claim 17 wherein the motion sensor and transmitter unit are positioned on the skin of the subject by the subject or under his or her direction.

20. The method as in claim 17 wherein said recording includes detecting spasmodic jerk of the xiphoid process in an anterior-posterior direction.

21. A method of quantifying cough frequency, comprising:
providing a motion sensor and a transmitter unit adapted for transmitting a bodily motion sensed by the motion sensor when in communication with the subject's body portion adjacent to the xiphoid process and diaphragm muscle; and, detecting the episodic movements of the xiphoid process in an anterior-posterior axis.
